(19) United States
(12) Patent Application Publication
(10) Pub. No.: US 2012/0046354 A1
(43) Pub. Date: Feb. 23, 2012

(54) ANTI-OXIDANT LOZENGES FOR THE PREVENTION AND TREATMENT OF RADIATION INDUCED MUCOSITIS, PRECANCEROUS LESIONS, ORAL CANCER AND OTHER ORAL CAVITY MUCOSAL DISORDERS

(76) Inventor: Eli D. Ehrenpreis, Skokie, IL (US)

(21) Appl. No.: 12/858,938

(22) Filed: Aug. 18, 2010

(51) Int. Cl. A61K 31/355; A61K 31/341; A61P 43/00; A61K 31/216; A61P 1/00; A61K 31/07; A61K 31/203

(52) U.S. Cl. 514/458; 514/725; 514/474; 514/559; 514/543

(57) ABSTRACT

A method for treating or preventing oral cavity mucosal disorders, comprising a step of providing a lozenge containing an antioxidant selected from the group of Vitamin A, Vitamin C, Vitamin E; and other ingredients and a step of placing the lozenge within the oral cavity for a period of time required for dissolution of the lozenge.

### FIELD OF THE INVENTION

The invention is directed to methods and compositions for preventing and treating oral cavity mucosal disorders. More particularly, the invention relates to methods and compositions for preventing and treating chronic oral cavity or oropharyngeal diseases using various forms of vitamin A in a lozenge form.

### BACKGROUND OF THE INVENTION

The primary functions of the mouth and throat are processing of food for digestion and swallowing these contents to move them into the digestive tract. The oral cavity has many functions, including sensation, mastication, secretion and provision of a barrier to the entry of toxins, carcinogenic substances and bacteria into the bloodstream.

The oral cavity is lined by squamous mucosa, similar to the skin. This lining is tightly attached to the mouth at the chewing or mascitatory portions of the cavity including the gingival and hard palate. Here the mucosa is keratinized. The floor of the mouth and buccal regions (the cheeks), are highly elastic and are covered with a nonkeratinizing epithelium. The distensibility of this portion of the oral cavity allows its many complex activities including speech and temporary storage of ingested material prior to the act of swallowing. The tongue has a different type of specialized epithelial lining.

The oral mucosa is characterized by rapidly proliferating precursor cells that continuously produce new epithelium, glandular cells for secretory functions and lymphoid tissue. A variety of inflammatory, ischemic infectious, traumatic and neoplastic disorders may affect the oral cavity. Radiation and chemotherapeutic agents damage oral epithelium, and destroy the ability of connective tissue to replace itself with regeneration. Ionizing radiation, often administered for the treatment of oral cancers, produces further damage by injuring ground substance. Radiation causes loss of polymerization of the ground substance, causing increased vascular permeability, edema and infiltration of inflammatory cells. A chronic condition characterized by fibrosis and damaged blood vessels results in tissue ischemia and hypovascularity. Mucosal atrophy and drying, ulceration, tissue friability and bleeding and secondary infections follow. Symptoms include oral and pharyngeal pain, dryness of the mouth, tongue and throat, difficulty swallowing (dysphagia), pain, bleeding malnutrition. Of interest, the pathophysiology of radiation-induced mucositis closely resembles radiation proctopathy, a form of injury of the rectum from radiation therapy administered for pelvic cancers such as cancers of the prostate, uterus and ovary. Our group has studied forms of vitamin A for treating radiation proctopathy.

Oral cancers develop in high risk patients following exposure to toxic substances such as alcohol, tobacco smoke and betel nuts. Patients with oral and head and neck cancers have a tendency to develop cancers throughout the oropharyngeal mucosa, suggesting that those at risk have oral and pharyngeal lining cells that are abnormal.

New research also suggests that the processes leading to the development of oral cancers is a step-wise one, beginning with precancerous changes in the oral cavity and oropharyngeal mucosa, making the development of agents that can potentially delay or prevent oral cancer possible.

### DETAILED DESCRIPTION OF THE INVENTION

Vitamin A has been demonstrated to accelerate wound healing following burn injury and surgeries in laboratory animals. The mechanism of this effect has not been fully determined, but increased cross linking of collagen and myofibrils occur after vitamin A administration. Beta carotene (a form of vitamin A) has also been shown to modify the effects of radiation and chemotherapy on their production of mucositis.

The inventor has described a dramatic case of the patient with AIDS and anal carcinoma who developed a large radiation-induced anal ulceration with marked debility. The patient required high dose opioid therapy for control of anal pain. After a twelve week course of orally administered vitamin A (in the form of retinyl palmitate), the patient experienced complete wound healing and symptomatic relief that persisted for more than six months. The inventor has also designed, conducted and published a randomized double-blinded trial comparing vitamin A in the form of retinyl palmitate (10,000 IU by mouth for 90 days) to placebo in patients with radiation proctopathy. The study determined that vitamin A significantly reduced rectal symptoms of radiation proctopathy, due perhaps to the wound healing effects of vitamin A.

The inventor has also tested a patient with a prior history of oral cancer that developed severe radiation-induced mucositis that was characterized by difficulty swallowing. This patient noted improvement in his condition following 4 weeks of treatment with retinyl palmitate lozenges.

Administration of vitamin A in a lozenge form should have efficacy for radiation damage and other chronic conditions of the mouth, since lozenges allow for the delivery of large concentrations of vitamin A directly at the affected area of the mouth. Additionally, retinyl palmitate is expected to reverse the damage to the lining of the mouth and oral organs caused by radiation therapy. The other form of vitamin A, beta carotene, when applied topically, could have the potential to enhance the effectiveness of retinol palmitate for treating radiation mucositis and other oral disorders.

Lozenges are bodies of solid materials into which one or several medications have been incorporated. These medications are then placed into the mouth, where they designed to dissolve and slowly release the medications that have been incorporated into the preparations. Medications within the lozenges are released at the site of placement, resulting in local effects of the medications. Lozenges often contain flavorings and/or sweeteners to make them more palatable for ingestion.

Oral lozenges of medications are available for placement in the mouth for the treatment of a variety of disorders. The most common use of oral lozenges is for the sore throat. Oral lozenges are also used to provide medications to geriatric patients and children that may have difficulty ingesting medications in standard forms. Lozenges come in hard, soft and chewable forms.

There are a variety of diseases of the oral cavity that may benefit from a lozenge containing topically applied vitamin A and anti-oxidant agents.

These conditions include (but are not limited to), damage from radiation therapy, mucositis from chemotherapeutic agents, Crohn's disease (oral ulcerations), other forms of mucositis, hyperplastic inflammatory lesions, erythematous macules and erosions, inflammatory hyperplasia, inflammatory granulomatous changes, apthous ulcers (canker sores), and other ulcerations, oral leukplakia, oral dysplasia, oral carcinoma in situ, oral condylomas, carcinoma and mucosal ischemia. These conditions represent a variety of significant clinical problems for which limited treatment options are currently available.

Oral cavity mucosal disorders are diagnosed by medical history, general physical examination, endoscopic oral and pharyngeal examination, CT scan and MRI.

There are a variety of treatment modalities for damage to the oral cavity from radiation and chemotherapy (mucositis). Supportive care makes up most of the treatments of mucositis. None of these treatment modalities have undergone rigorous scientific evaluation although some of these treatments have demonstrated efficacy when small studies on their use have been performed.

Initially, patients are recommended to undergo general dental care to maintain cleanliness of the mouth and gums. Repeated cleansing of the oral cavity is suggested.

Mouthwashes containing anti-inflammatory medications such as benzydamine hydrochloride have been employed. This is an anti-inflammatory agent that may also eliminate some oral bacteria. Other topical treatments for mucositis include corticosteroids to reduce inflammation, and chamomile. Antiseptic solutions such as chlorhexidine, in the form of a mouthwash, are often recommended for mucositis. Studies seem to indicate that chlorhexidine has limited or possibly no effectiveness for this condition.

Other practitioners have tried using mouth rinses with pavodine and hydrogen peroxide. These are unstudied treatments. Antibiotic therapy may have a role in treating mucositis. An antibiotic lozenge containing polymixin E, tobramicin and amphotericin B may prevent mucositis when used by patients that are receiving radiation therapy. Anaesthetic agents when given topically can reduce discomfort associated with mucositis. These include viscous lidocaine and dyclonine. Many practitioners prescribe a “magic mouthwash,” of topical treatments for oropharyngeal pain. A typical example may include viscous lidocaine, diphenhydramine, dexamethasone, topical, antibacterial antibiotics, antifungal antibiotics, Maalox, sorbitol, and flavoring. Potent local anesthetic applications, including viscous lidocaine with 1% cocaine or dyclonine, may work at the expense of loss of the sensation of swallowing. Narcotics are prescribed for severe pain, but these have a drying effect on the saliva and on mucous secretion. Vitamin E, an antioxidant, may be beneficial in treating mucositis. Topical vitamin E has been used to treat mucositis after chemotherapy. A single study of 18 patients found benefit of 1 mL of topical vitamin E (400 mg) oil compared to soybean oil applied to mucosal lesions twice daily. Topical sucralfate, prostaglandin and allopurinal may be beneficial in preventing the occurrence of mucositis in patients undergoing treatment with chemotherapeutic drugs.

Of interest, orally administered beta carotene, a form of vitamin A, may decrease the likelihood of the development of mucositis following chemotherapy and radiation therapy. Mills conducted a study of 20 patients with oral squamous cell carcinoma who received 30 daily fractions of telecobalt radiation therapy. This was given in combination with vincristine, bleomycin, methotrexate, and leucovorin. Patients received a standard diet with or without beta-carotene after randomization.

Dosage of beta-carotene initially was 250 mg daily for 21 days followed by 75 mg daily for the remaining time that they received treatment. The treatment group had 22 patient-weeks of severe mucositis compared with 38 patient-weeks in the control group (P=0.025). At present, topical vitamin A or beta carotene have not specifically been studied as treatments for radiation or chemotherapy induced mucositis.

Other treatments that are being investigated include laser treatment with low energy lasers, silver nitrate and glutamine. None of these treatments are proven effective.

Thus, there is a need to develop methods and compositions that may be used to prevent and treat radiation and chemotherapy-induced mucositis as well as other oral cavity mucosal disorders. Ideally, identification of new agents that may also alter the pathophysiology of oral cavity mucosal disorders is suggested.

Chemoprevention is a proposed method to use in patients that are at high risk for the development of oral cavity or oropharyngeal cancer. This method employs a drug or chemical to treat premalignant lesions (such as leukoplakia) or keep these lesions from progressing into cancers. Synthetic retinoids (vitamin A derivatives) have been studied as treatment for premalignant oral cavity or oropharyngeal lesions. The retinoids studied for these conditions are 13-cis-retinoic acid, all-trans-retinoic acid, and etretinate; all were active with overall response rates of 59 to 92 percent. A randomized, placebo-controlled study of isotretinoin (13-cis-retinoic acid, 1 to 2 mg/day) was performed in 44 patients with leukoplakia by Hong, et al [46]. Following three months of therapy, the clinical response rates (67 versus 10 percent) and the response rates among patients with biopsy-proved dysplasia (54 versus 10 percent) were significantly higher in the patients treated with isotretinoin compared to placebo. This effect may be mediated by down regulation of nuclear retinoid receptor beta (RAR beta), a component of several bipartite transcription factors that respond to treatment with retinoic acid. This treatment was limited by side effects. For example, 47 percent of patients treated with higher dose isotretinoin (2 mg/kg per day) required decreases in their dosage. Although the lower dose of 1 mg/kg per day was less toxic, xeroderma was common and 29 percent of patients developed conjunctivitis. Unfortunately, about half of the patients had a relapse of disease within three months of discontinuation of treatment. No studies of isotretinoin administered in a lozenge form to treat or decrease the occurrence of oral cavity or oropharyngeal cancer or to treat oral leukoplakia have previously been performed. However, ONYX-015, a medication containing the p53 tumor suppressor gene has been administered in the form of a mouthwash to patients with oral leukoplakia. Following up to 12 months of treatment, approximately 32% of patients in the study had remission of their oral leukoplakia. On the other hand, topically applied ketolorac, a nonsteroidal anti-inflammatory agent did not appear to effectively treat oral leukoplakia.

The present invention provides a method for preventing and treating oral cavity or oropharyngeal mucosal disorders. The method includes a step of providing a lozenge containing between 100 and 50,000 IU of retinyl palmitate. The lozenge will be administered daily or up to 6 times daily. Lozenges can be for short-term use such as one to seven days. Alternatively, Lozenges may be used on a regular basis, including long-term daily use up to six times daily, depending on the condition being treated. Other ingredients such as antioxidants including beta carotene (100 to 50,000 IU), Vitamin C (25-500 mg), and Vitamin E (100-1000IU) may be included. The lozenge may also contain other synthetic retinoids including 13-cis-retinoic acid (also known as isotretinoin), (1-100 mg), all-trans-retinoic acid (also known as tretinoin), (0.1-10 mg), and etretinate (0.025-0.33 mg).

The method further includes a step of placing the lozenge within the oral cavity for a period of time required for dissolution of the lozenge, especially in patients with xerostomia. A local spray or other forms for direct application are considered.

Also disclosed is an oral lozenge or troche for use in treating oral cavity mucosal disorders. The lozenge includes a soluble base; and an ingredient selected from the group of retinyl palmitate, Vitamin A, Vitamin C, Vitamin E and other ingredients including antioxidants and other synthetic retinoids encapsulated within the soluble base.

According to a preferred embodiment, the form of vitamin A contained within the oral lozenge is retinyl palmitate provided at a dosage of between 100 and 50,000 IU.

According to a further aspect of the invention, the oral lozenge may further include an antioxidant selected from the group of Beta carotene, Vitamin C, and Vitamin E and others such as green tea, etc.

According to a further aspect of the invention, the oral lozenge may further include synthetic retinoids such as -cis-retinoic acid, all-trans-retinoic acid, and etretinate.

According to yet another aspect of the invention, the oral lozenge may further include additional agents that may prevent and/or treat mucositis. These treatments may include anti-inflammatory agents, antibiotics, antiseptics, sucralfate, sodium alginate, antacids, diphenhydramine, kaolin and pectin, prostaglandin E2, allopurinol, sorbitol, pentoxyphylline, polyethylene glycol and/or anaesthetic agents such as lidocaine and tronilaine.

A variety of chronic, painful and debilitating disorders may affect the oral cavity and pharynx. Examples of these disorders include mucositis, dysplastic and neoplastic lesions and inflammatory disorders.

Radiation and chemotherapy induced mucositis, a consequence of prior radiation for oral cavity or oropharyngeal cancers causes symptoms due to damage and stiffness of the lining of the oral cavity and bleeding from decreased blood flow, tissue fibrosis and new blood vessels that form as a response to decreased local blood flow.

Neoplasms are abnormal growths of the oral region and may cause pain, ulcerations and bleeding. Oral cancer generally occurs in patients who consume large quantities of alcohol in combination with tobacco abuse. Human Pappiloma Virus (HPV) and other viruses may be additional cofactors. Longstanding damage to the oral mucosa from irritation from tobacco, exposure to carcinogens and inflammation secondary to infection may cause dysplasia which can degenerate into oral cavity or oropharyngeal cancer. Field effects and step-wise carcinogenesis that have demonstrated to occur in the setting of these cancers provide opportunity for chemoprevention in patients with precancerous oral cavity or oropharyngeal lesions such as leukoplakia as well as other high risk patient groups.

The present invention provides an inexpensive, innovative method for preventing and/or treating these conditions in mammals, including humans. The technique of the present invention is a substitute or adjunct for conventional treatments for these disorders.

The device of the present invention provides an alternative to the conventionally used therapies for these conditions when available, and provides a new opportunity for prevention and treatment when no current therapies are available.

Vitamin A, a fat soluble vitamin, is present in pigmented vegetables and animal tissues. It is an important factor in growth of the epithelium, bone and retina. Vitamin A is composed of retinyl palmitate and beta carotene.

Vitamin A has been demonstrated to have a number of beneficial qualities and is used as medical therapy for a variety of conditions. Vitamin A has been shown to enhance the function of the immune system, accelerates the rate of wound healing and may be used as treatment for precancerous conditions.

Administration of vitamin A, other anti-oxidants, other retinoids and other medications in a lozenge form (or other topical forms) has the following potential advantages: 1) Larger doses will be delivered directly to the diseased area, resulting in more potent, better therapy for the conditions. 2) The amount of the delivered substance will undergo much less absorption into the systemic circulation. For certain substances, potent doses of the substance will be directed to the effected areas while fewer toxic effects of the compound are expected to occur.

In a preferred embodiment, vitamin A in the form of retinyl palmitate (with or without other forms of vitamin A including beta carotene) is incorporated into a lozenge for application into the mouth for the treatment of oral cavity mucosal disorders. This treatment is administered in a lozenge form for patients suffering from chronic oral cavity mucosal disorders. These treatments may be administered on a daily basis, or possibly more frequently.

In an alternate use of the invention, vitamin A lozenges (including a variety of potential additional ingredients) may be administered to patients at high risk (such as tobacco users) to prevent the development of oral dysplasia and oral cancer, especially after treatment for a first cancer. Additionally, vitamin A lozenges would be administered to patients with oral leukoplakia or dysplasia to prevent the development of oral cancer. Lozenges may be taken at the time when radiation and/or chemotherapy are administered to prevent the development of mucositis or limit the severity of the condition, or after treatment with radiation or chemotherapy to treat chronic side effects.

Lozenges containing other anti-oxidants, other retinoids and other medications in a lozenge form are also proposed for the above uses.

Production of lozenges

Lozenges are compounded to have the properties of slow oral disintegration. Because of this, they are often used for direct effects in the mouth. Other methods of direct application are available as well. Commonly, the active ingredients of oral lozenges include local anesthetics and antiseptics including phenol, sodium phenolate, benzocaine, and cetylpyridinium chloride. Sweeteners and flavorings are also added to enhance the palatability of oral lozenges. Lozenges maintain contact of the drug in contact with the lining of the mouth and throat for extended lengths of time. The following is a summary of manufacturing and compounding issues related to the production off lozenges. This information was obtained from a website on the Compounding Lab maintained by the University of North Carolina, School of Pharmacy. The web address for this website is http://pharmlabs.unc.edu/labs/lozenge/lozenges.htm.

Lozenges can be made by molding or by compression. The name troche is applied to compressed lozenges. But in lay language, lozenge and troche are used interchangeably. Commercial lozenges are made by compression; they are harder than ordinary tablets so they will slowly dissolve or disintegrate. Compounded lozenges can be prepared by molding mixtures of ingredients containing:
sugars to form a hard lozenge
polyethylene glycol (PEG) to form a soft lozenge
gelatin to form a chewable lozenge

Hard lozenges might be considered solid syrups of sugars. These dosage forms are made by heating sugars and other ingredients together and then pouring the mixture into a mold. The molds can shape the mixture to look like a sucker or a lollipop. The hard lozenge will not disintegrate in the mouth but will erode or dissolve over a 5-10 minutes period. Hard lozenges are similar to hard candy. In fact, many hard lozenge formulas are modifications of hard candy formulas. The dosage form needs a low moisture content (0.5-1.5%) so water is evaporated off by boiling the sugar mixture during the compounding process. It is critical to follow all formulation instructions carefully and pay particular attention to temperatures; the success of forming the amorphous crystalline produce depends on it. For example, if a formula states that the syrup should not be stirred until a particular temperature is reached, or if it states that the temperature of the syrup must reach 145° C., follow these instructions precisely.

The primary disadvantage of hard lozenges is apparent. Heat labile drugs cannot be used in this formulation because of the high temperatures required for preparation. Another problem is that hard lozenges become grainy. The speed at which this happens depends on the sugar used and its concentration in the formulation. The best compromise seems to be 55-65% sucrose and about 35-45% corn syrup.

The final pH of the product should be considered. If acidic flavoring agents are to be used (citric, tartaric, fumaric acid), the final pH may be quite low (around 3). Hard candies have a pH around 5 to 6. So the pH of the hard lozenge will need to be raised to obtain this range. Calcium carbonate, sodium bicarbonate, and magnesium trisilicate can be added to the formulation for this purpose.

Soft lozenges are easily compounded and can be colored and flavored. They can either be chewed or allowed to slowly dissolve in the mouth. They are typically made of ingredients such as polyethylene glycol (PEG) 1000 or 1450 (low molecular weight), chocolate, or a sugar-acacia base. Because of their soft texture, these lozenges can be hand rolled and then cut into pieces which contain the correct amount of active ingredient. But a more convenient dispensing method is to pour the warm mass into a plastic troche mold.

Start the pour in the B2 position and then continue to fill all of the cavities in one pouring. PEGs contract as they cool so the cavities of the troche mold should be overfilled. Chocolate does not shrink as it cools so overfilling is not necessary. Before the material has solidified in the mold, use the edge of a spatula to level and even out the poured mass. Once the material has cooled in the troche mold, a warm spatula or light heating with a hot air blow dryer can give a smooth appearance.

Some soft lozenge formulations contain acacia and silica gel. Acacia is used to add texture and smoothness to the lozenge and silica gel is used as a suspending agent to keep materials from settling to the bottom of the mold cavity during the cooling process. Mixtures are typically heated to about 50° C. during preparation so other ingredients such as sweeteners (i.e., sodium saccharin) should be heat stable.

Flavoring and sweetening soft lozenges to achieve a satisfactory taste will take some experimentation on the part of the pharmacist. Flavors can be obtained from sources such as food extracts, syrup flavor concentrates, or volatile oils. Sweeteners, either alone or in combination, will need to be added. A mixture of 9 parts Nutrasweet® and 1 part saccharin might be a suitable starting point for developing a suitable sweetener.” Chewable lozenges are popular with the pediatric population since they are “gummy-type” lozenges. These lozenges are often highly fruit flavored and may have a slightly acidic taste to cover the acrid taste of the glycerin. Again, experimentation will be needed to achieve a satisfactory flavored and sweetened formulation. If the lozenge is to be used to enhance buccal or sublingual absorption, then a gelatin soft lozenge would be a good base since it dissolves slower than the polyethylene glycol bases. More drug would be absorbed from the buccal cavity and less would be swallowed and lost in the GI tract. But the long contact time would limit gelatin's usefulness if the drug is extremely bitter or if the taste is hard to mask. The polyethylene glycol bases are more palatable for the patient and should be used if buccal or sublingual absorption is not a therapeutic goal.”

Another potential production method under consideration is to melt commercially available lozenge-like candies using a low heat until a fluid is obtained, incorporate the drug into the liquid mixture, and then cool the mixture.

The term “vitamin A” refers to the variety of chemicals having the same properties in the living organism as retinol. This term also includes specific chemicals that include retinol and retinol esters, as well as retinoids and chemical analogues from the retinoid family.

The terms “oral” and “oropharynx” refer to the anatomic body structures making up the mouth and the throat.

By “lozenge,” the invention includes the production of a solid substance that is administered into the mouth that contains medication for direct delivery to the lining of the oral cavity.

The term “oral cavity and oropharyngeal mucosal disorders” refers to the group of abnormal conditions occurring in humans or animals that produce characteristic symptoms in the mouth and throat and are associated with appropriate findings on physical examination and endoscopy.

“Endoscopy” is a diagnostic tool utilized to examine the lining of the oral cavity and the gastrointestinal tract. In this setting, endoscopy of the mouth and throat is performed.

The term “mucositis” is used in the invention refers to inflammation of the lining of the mouth and throat.

The term “dysplasia” refers to premalignant or precancerous changes occurring in a body tissue.

The term “neoplasia” refers to abnormal cell growth that may be benign or malignant.

The term “leukoplakia refers to a precancerous lesion of the oral and pharyngeal mucosa.

The term “IU” refers to international units of measure.

The invention provides methods and compositions for the treatment of oral cavity mucosal disorders.

More specifically the present invention discloses a lozenge base containing vitamin A in various forms including anti-oxidants that is placed in the mouth of a patient for a specific time period that allows for the treatment and/or prevention of chronic oropharyngeal diseases. The use of a lozenge to deliver treatment, facilitates a more direct delivery of treatment to the affected region(s) of the body, and mitigates the toxicity problem associated with absorption of high amounts of the ingredients of the treatment.

According to one embodiment, the lozenge contains between 100 and 50,000 IU of vitamin A in the forms of retinyl palmitate and/or beta carotene. The lozenge may be constructed from a variety of bases as previously described.

The lozenge containing vitamin A and other ingredients is taken orally to reside within the oral cavity for the time period required for dissolution. Since the lozenges are fully dissolvable, release of vitamin A from the lozenges will be achieved after residence of the lozenge in the mouth. The released vitamin A will occur in high concentrations at the site of delivery, thus enhancing the effectiveness of this therapy for oral cavity mucosal disorders.

According to an alternative embodiment, the lozenges may include other retinoids.

According to an alternative embodiment, the lozenges may include other antioxidants such as vitamin E and vitamin C and natural antioxidants such as fish oils, green tea, cranberry, etc. These may be used as distinct lozenge preparations or as additional components of the vitamin A lozenges.

According to an alternative embodiment, the lozenges may be manufactured by substituting low molecular weight PEG for standard weight PEG.

According to another aspect of the invention, the oral lozenge may further include additional treatments for mucositis. These treatments may anti-inflammatory agents, antibiotics, antiseptics, sucralfate, sodium alginate, antacids, diphenhydramine, kaolin and pectin, prostaglandin E2, allopurinol, sorbitol, pentoxyphylline, polyethylene glycol and/or anaesthetic agents such as lidocaine and tronilaine.

These uses and in any of the embodiments of the invention, a lozenge form of these agents are used as a clinical treatment for chronic diseases of the mouth and throat. These same ingredients may alternatively be included in other forms of topical treatments of the mouth and throat including local sprays, gels or liquids.

Any form of vitamin A that is placed in a lozenge form for the treatment of oral cavity mucosal disorders is within the confines of the invention. Additionally, the incorporation of vitamin A in combination with other antioxidants into lozenges technique to treat oral or oropharyngeal diseases is embodied within this invention. Additionally, the incorporation of vitamin A derivatives in the form of retinoids alone or in combination with vitamin A and other antioxidants into lozenges as a technique to treat oral or oropharyngeal diseases is embodied within this invention. Additionally, the incorporation of any antioxidant substances within the lozenges, utilizing this technique as a means of treating oral cavity mucosal disorders is embodied in the invention. Additionally, incorporation of low molecular weight PEG into the ingredients of the lozenge is within the confines of the invention. Finally, other agents, such as anti-inflammatories, anaesthetics, herbals or other vitamins may be included in the lozenges to enhance the efficacy of the vitamin A.

Lozenges may of the hard, soft or chewable.

In particularly preferred embodiments, the medication contained in the lozenge is vitamin A. In the preferred embodiment, the lozenge is composed of typical hard, soft or chewable bases. However, other bases may be employed in the invention to allow for direct application of the medication to the lining of the mouth and throat. Low molecular weight PEG may be substituted for standard PEG in the production of the lozenges. More generally, any form of lozenge base may be used to construct the devise. In addition, other retinoids as well as a variety of antioxidants and other substances may be incorporated into the lozenge to allow direct application of these substances to the lining of the mouth and throat. Other aforementioned substances may also be included in the lozenges to enhance their efficacy in the treatment or prevention of oropharyngeal disorders. The contents of the lozenge may also consist of a variety of antioxidants, either alone or in combination with vitamin A, depending on the goal of treatment.

Variable doses of vitamin A and other ingredients may be utilized, depending on the condition being treated. Ranges of doses of vitamin A and retinoids are listed above. Both lower and higher doses than these would initially be employed in the construction of the devise. The optimal dosage to treat these conditions will be determined based on further evaluation.

However, following appropriate clinical evaluation of this treatment, either larger or smaller doses of vitamin A may ultimately be used for treating radiation and chemotherapy induce mucositis as well as other oral cavity mucosal disorders.

Dosing for vitamin A, other retinoids and other medications in a lozenge form and other antioxidants in lozenges are anticipated to be less than oral doses for the treatment and/or prevention of oral or oropharyngeal diseases, since these agents will be directly applied to the affected areas. However, higher doses may also be studied and utilized based on further clinical trials.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. A lozenge for preventing and treating oral cavity mucosal disorders, comprising Vitamin A provided at a dosage of between 100 and 50,000 IU.

2. The lozenge of claim 1, further comprising a one or more ingredients from the group of Vitamin C, and Vitamin E.

3. The lozenge of claim 1, further comprising an anti-inflammatory agent.

4. The lozenge of claim 1, further comprising an anaesthetic agent.

5. The lozenge of claim 1 further comprising synthetic retinoids.

6. The lozenge of claim 1 further comprising 13-cis-retinoic acid.

7. The lozenge of claim 1 further comprising all-trans-retinoic acid,

8. The lozenge of claim 1 further comprising etretinate.

9. A method for preventing and treating oral cavity mucosal disorders, comprising orally administering a lozenge having Vitamin A provided at a dosage of between 100 and 50,000 IU.

10. The method of claim 9, wherein the lozenge further includes one or more ingredients from the group of Vitamin C, and Vitamin E.

11. The method of claim 9, wherein the lozenge further includes an anti-inflammatory agent.

12. The method of claim 9, wherein the lozenge further includes an anaesthetic agent.

13. The method of claim 9, wherein the lozenge includes vitamin A derivatives in the form of retinoids alone or in combination with vitamin A.

14. The method of claim 13 wherein the lozenge further comprises other antioxidants .

15. The method of claim 9, wherein the lozenge includes other synthetic retinoids.

16. The method of claim 9, wherein the lozenge includes between 1-100 mg 13-cis-retinoic acid.

17. The method of claim 9, wherein the lozenge includes between 0.1-10 mg all-trans-retinoic acid.

18. The method of claim 9, wherein the lozenge includes between 0.025-.33 mg etretinate.
